(19) United States
(12) Patent Application Publication
(10) Pub. No.: US 2010/0228175 A1
(43) Pub. Date: Sep. 9, 2010

(54) SYSTEM FOR HARVESTING AND DISPENSING BLOOD CLOT

(76) Inventors: Javin C. Pierce, Stowe, VT (US); Peter Kurzweil, Huntington Beach, CA (US)

Correspondence Address: Mark J. Pandiscio;Pandiscio & Pandiscio, P.C., 470 Totten Pond Road, Waltham, MA, 02451, US

(21) Appl. No.: 12/714,047

(22) Filed: Feb. 26, 2010

Related U.S. Application Data
(60) Provisional application No. 61/155,842, filed on Feb. 26, 2009.

(51) Int. Cl. A61M 37/00

(52) U.S. Cl. 604/4.01

(57) ABSTRACT

A system for harvesting and dispensing blood clot, the system comprising:

apparatus for drawing blood;

apparatus for stirring the blood so as to form clot; and

apparatus for delivering clot to a body.

A method for harvesting and dispensing blood clot in a patient, the method comprising:

withdrawing blood from the patient;

forming clot;

removing clot; and

delivering clot into the patient.

### REFERENCE TO PENDING PRIOR PATENT APPLICATION

This patent application claims benefit of pending prior U.S. Provisional Patent Application Ser. No. 61/155,842, filed Feb. 26, 2009 by Javin C. Pierce et al. for SYSTEM FOR HARVESTING AND DISPENSING BLOOD CLOT (Attorney's Docket No. PIERCE-3 PROV), which patent application is hereby incorporated herein by reference.

### SUMMARY OF THE INVENTION

A blood clot harvesting and dispensing system is provided that serves to augment surgical tissue repair. Blood clots form naturally to staunch bleeding, and the platelets also serve to carry and deposit growth factors that aid tissue healing at the damaged site. The invention comprises a means to draw blood, a means to precipitate clot from blood, and a means to reliably dispense clot in a controlled manner into the body. The invention also comprises a method and apparatus for placement of clot directly into tendon. The invention also comprises a means to harvest growth factors from blood and deposit them in a tissue defect to promote healing.

In one preferred form of the invention, there is provided a system for harvesting and dispensing blood clot, the system comprising:

apparatus for drawing blood;

apparatus for stirring the blood so as to form clot; and

apparatus for delivering clot to a body.

In another preferred form of the invention, there is provided a method for harvesting and dispensing blood clot in a patient, the method comprising:

withdrawing blood from the patient;

forming clot;

removing clot; and

delivering clot into the patient.

### BRIEF DESCRIPTION OF THE DRAWINGS

These and other objects and features of the present invention will be more fully disclosed or rendered obvious by the following detailed description of the preferred embodiments of the invention, which are to be considered together with the accompanying drawings wherein like numbers refer to like parts, and further wherein:

FIG. 1 illustrates a system for harvesting and dispensing blood clot.

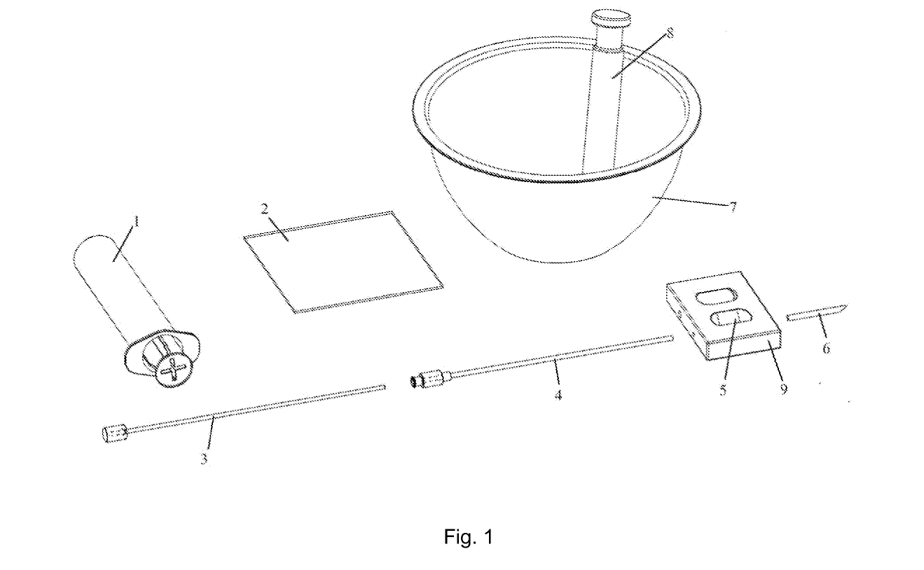

### DETAILED DESCRIPTION OF THE PREFERRED EMBODIMENTS

The preferred embodiment of the invention comprises a conventional blood drawing needle and flexible tubing, a syringe (1), a bowl or vessel (7), and a frosted glass rod (8) for stirring the blood for 3-10 minutes until a clot forms. The loader (9) comprising funnel or “hopper”(5) receives the clot, a hollow shaft instrument (4) that interacts with the loader guide, in said funnel or “hopper”, said hollow shaft instrument having a thin rim capable of cutting morsels of clot when pushed against a flat surface at the end of the loader guide, and a plunger (3) to expel the clot into the body.

Among other things, the invention comprises:
A. (1) a means to draw blood;
(2) a means to precipitate clot from blood; and
(3) a means to reliably dispense clot in a controlled manner into the body.
B. The invention of (A) wherein (2) is a frosted glass rod that is stirred manually or by a motor in a container preferably of metal.
C. The invention of (A) wherein (2) is a metal member with a highly disrupted surface (such as deep scratches, hole, screen, etc.).
D. The invention of (A) wherein (2) comprises an anodic electrode for precipitation of clot, and a cathode.
E. The invention of (D) wherein dissimilar metals are employed to provide anodic precipitation of clot onto a collector.
F. The invention of (A) wherein (3) comprises a loader having a funnel shape, or “hopper” to facilitate loading of the clot into the dispenser.
G. The invention of (F) wherein the loader comprises a coacting guide means with the dispenser to facilitate placement of suture through the clot.
H. The invention of (F) wherein (3) comprises a hollow instrument that receives the clot and dispenses it into the body.
I. The invention of (H) wherein (3) comprises a plunger that coacts with the hollow dispenser to extrude clot.
J. The invention of (I) wherein a suture passer coacts with the hollow clot dispenser to pass a suture through the clot.
K. The invention of (F) wherein suction is applied to the dispenser to draw clot into it.
L. The invention of (K) wherein clot is dispensed into the body via pressure with a syringe.
M. The invention of (H) wherein the dispenser has a sharp edged rim suitable for morselizing segments of clot against a flat surface, thereby packing clot into the dispenser.

The optional steps to utilize the invention with a suture loading technique are:

Stir blood until it clots on frosted plunger.

Remove clot with gauze and blot off excess liquid.

Place clot into hopper.

Place coring tube into block until end is touching round well.

Force clot deep into well with gauze protected thumb.

Advance coring tube to end of well and twist back and forth to chop off a core.

The white guide tube (on the purple passer) can be placed into coring tube and will back out as tube gets filled.

When desired amount of clot is filling tube, run the sharp passer through the breech of the coring tube (while it is still in block) until it hits the end.

Back out the coring tub to edge of well and clear well of excess clot.

Place a corner of gauze deep into well and advance the passer through the gauze till it hits the end again.

Retract the coring tube and the passer tip will be proud of the end of the coring tube and clot inside it.

Deploy wire loop and load suture into it and drag suture through the tube and clot while holding the white guide tube.

Remove white guide tube and replace it with the white plunger with small black tip.

Suture will be though middle of clot and clot can be extruded with plunger.

### MODIFICATIONS

It will be appreciated that still further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. A system for harvesting and dispensing blood clot, the system comprising:
apparatus for drawing blood;
apparatus for stirring the blood so as to form clot; and
apparatus for delivering clot to a body.

2. A method for harvesting and dispensing blood clot in a patient, the method comprising:
withdrawing blood from the patient;
forming clot;
removing clot; and
delivering clot into the patient.
